(19) United States
(12) Patent Application Publication
(10) Pub. No.: US 2025/0259568 A1
(43) Pub. Date: Aug. 14, 2025

(54) ANATOMICAL MODELS AND ASSOCIATED METHODS FOR EVALUATING COMPRESSION

(71) Applicant: ARTHREX, INC., Naples, FL (US)

(72) Inventors: Nick Metcalfe, Bonita Springs, FL (US); David Knopf, Bonita Springs, FL (US); Benjamin David Bernarding, Allison Park, PA (US); David Yang, Bedord, MA (US); Shervin F. Kazemi, Naples, FL (US); Gage Weimer, Ruskin, FL (US); Maris V. Prieditis, Naples, FL (US)

(21) Appl. No.: 19/048,420

(22) Filed: Feb. 7, 2025

Related U.S. Application Data
(60) Provisional application No. 63/551,574, filed on Feb. 9, 2024.

(51) Int. Cl. G09B 23/34; A61B 17/00; A61B 17/72

(52) U.S. Cl. CPC G09B 23/34; A61B 17/72; A61B 2017/00716

(57) ABSTRACT

This disclosure relates to surgical systems, devices and methods for planning and implementing surgical procedures. The systems and methods disclosed herein may be utilized to establish physical anatomical models of anatomy. A compression feature may be utilized to evaluate compression between portions of the physical anatomical model.

### CROSS-REFERENCED TO RELATED APPLICATION

This application claims priority to U.S. Provisional Application No. 63/551,574, which was filed on Feb. 9, 2024.

### BACKGROUND

This disclosure relates to surgical systems, devices and methods for planning and implementing surgical procedures utilizing physical models of anatomy.

Deformities may form along various bones and joints of the human musculoskeletal system. Surgeons may prepare for surgery by performing a procedure on a cadaveric or saw bone specimen.

### SUMMARY

The systems and methods disclosed herein may be utilized to establish physical anatomical models of anatomy. A compression feature (e.g., spacer) may be utilized to evaluate compression between portions of the physical anatomical model.

A system for an orthopaedic procedure according to an implementation may include a physical anatomical model including a main body representative of anatomy. The main body may include a first bone portion and a second bone portion. A compression spacer may be positionable to establish a gap at an interface between the first bone portion and the second bone portion. The compression spacer may be associated with a preselected compressive force relating to the gap.

A system for an orthopaedic procedure according to an implementation may include a physical anatomical model representative of anatomy. The physical anatomical model may include a first bone portion and a second bone portion. A compression spacer may be dimensioned to separate the first bone portion and the second bone portion under a compressive load, such that removal of the compression spacer may cause movement between the first bone portion and the second bone portion.

A method of rehearsing for a surgical procedure according to an implementation may include accessing a virtual anatomical model associated with an anatomy of a patient. The method may include generating a configuration associated with a physical anatomical model that may be representative of the virtual anatomical model. The method may include forming the physical anatomical model. The physical anatomical model may include a body that may be associated with an anatomical profile of a bone. The body may include a first bone portion and a second bone portion. The method may include forming a compression spacer that may be insertable between the first bone portion and the second bone portion.

A compression spacer for a surgical procedure according to an implementation may include a spacer body configured to be received between first and second bone portions of a physical anatomical model. The spacer body may be dimensioned to have a thickness associated with a compressive force of an implant associated with abutment of the first and second bone portions.

These and other features may be best understood from the following specification and drawings, the following of which is a brief description.

### BRIEF DESCRIPTION OF THE DRAWINGS

FIG. 1 discloses a planning system according to an implementation.

FIG. 2 discloses another implementation of a planning system including a user interface.

FIG. 3 discloses the user interface of FIG. 2 including display windows depicting adjacent bone models.

FIG. 4 discloses a system for a surgical procedure according to an implementation.

FIGS. 5A-5C disclose a compression spacer relative to the system of FIG. 4.

FIG. 6 discloses a view of the system of FIG. 4 with the compression spacer removed.

FIGS. 7A-7C disclose views of the system of FIG. 4 at different orientations.

FIG. 8 discloses a cross-sectional view of the system of FIG. 4.

FIG. 9 discloses an implementation of the system in FIG. 4, in which the compression spacer is removed and sufficient compressive force may be achieved.

FIG. 10 discloses an implementation of the system in FIG. 4, in which the compression spacer is removed and insufficient compressive force may be achieved.

FIG. 11 discloses an implementation of the system in FIG. 4, in which a locking key is provided within the first and second bone portions.

FIG. 12 discloses an example locking key.

FIG. 13 discloses a system for a surgical procedure according to another implementation.

FIG. 14 discloses a method of planning and implementing a surgical procedure utilizing physical anatomical model(s).

FIG. 15 discloses a technique for forming a physical anatomical model.

Like reference numbers and designations in the various drawings indicate like elements.

### DETAILED DESCRIPTION

This disclosure relates to surgical systems, devices and methods for planning and implementing surgical procedures utilizing physical models of anatomy. Physical anatomical models may be utilized to rehearse and train for various surgical procedures.

The disclosed techniques may be utilized to provide the surgeon a preoperative planning and/or training experience that may be targeted or tailored to the surgeon based on procedure, skill set, experience, etc. The surgeon may select a particular configuration of a virtual anatomical model that may be fabricated or otherwise formed to establish a physical anatomical model based on the anatomy or pathology that the surgeon may intend to treat. The surgeon may utilize the physical anatomical model to train with particular instrumentation, implants and other devices that may be intended for a planned surgery to treat a patient.

The surgeon, assistant or other user may interact with a graphical user interface (GUI) to select various parameters or characteristics of the physical anatomical model. The parameters may include anatomy, patient, fracture classification, case, etc., to establish a desired configuration of the physical anatomical model. The specified parameters may be represented in the physical anatomical model.

Various techniques may be utilized to establish the physical anatomical models, including any of the techniques disclosed herein. A compression feature may be utilized to evaluate compression between portions of the physical anatomical model. In implementations, the compression feature may include a compression spacer. The compression spacer may be dimensioned and formed to evaluate the compression associated with an implant. The compression spacer may be dimensioned to be situated between two (e.g., adjacent) bone portions of the physical anatomical model. The spacer may then be removed to evaluate compression between the two bone portions, such as the compressive force established by an implant that may extend between the two portions. In implementations, a set of compression spacers of various dimensions (e.g., thicknesses) may be provided with the physical anatomical model as a kit. The surgeon or clinical user may select one or more compression spacers from the kit to evaluate the compression associated with a selected implant that may be utilized to secure the two portions to each other.

The repaired physical anatomical model may serve as an artifact for the surgeon. The surgeon may leave a training facility with a revised physical anatomical model once training is completed. The surgeon may refer to the revised physical anatomical model prior to and/or during a surgical procedure on a respective patient.

A system for an orthopaedic procedure according to an implementation may include a physical anatomical model including a main body representative of anatomy. The main body may include a first bone portion and a second bone portion. A compression spacer may be positionable to establish a gap at an interface between the first bone portion and the second bone portion. The compression spacer may be associated with a preselected compressive force relating to the gap.

In any implementations, the compression spacer may include a spacer body extending between a first surface and a second surface. The first surface may be dimensioned to abut the first bone portion. The second surface may be dimensioned to abut the second bone portion.

In any implementations, the spacer body may include a periphery adjoining the first surface and the second surface.

In any implementations, the periphery of the spacer body may have a contour dimensioned to substantially match a bone contour of at least one of the first bone portion and the second bone portion.

In any implementations, the compression spacer may include a slot dimensioned to allow passage of an implant through the interface.

In any implementations, the compression spacer may include a first protrusion receivable in a first opening in the first bone portion to limit relative movement between the compression spacer and the first bone portion.

In any implementations, the first protrusion may include a cylinder having a longitudinal axis that may be substantially parallel with the first surface.

In any implementations, the compression spacer may include a second protrusion receivable in a second opening in the first bone portion.

In any implementations, the first protrusion may be opposite the slot from the second protrusion.

In any implementations, the first protrusion may extend from the first surface, the first opening may be established in the first bone portion, the second protrusion may extend from the second surface, and the second opening may be established in the second bone portion such that the compression spacer may limit relative movement between the first and second bone portions at the interface in an assembled position.

In any implementations, the compression spacer may include a first protrusion receivable in a first opening in the first bone portion to limit relative movement between the compression spacer and the first bone portion.

In any implementations, the compression spacer may include a second protrusion receivable in a second opening in the second bone portion to limit relative movement between the compression spacer and the second bone portion.

In any implementations, the compression spacer may include an aperture dimensioned to receive an implant extending through the first bone portion and the second bone portion.

In any implementations, the implant may include an intramedullary nail.

In any implementations, the preselected force may be selected such that removal of the compression spacer may cause the first and second bone portions to abut each other at the interface. The compression spacer may be removable to evaluate compression between the first and second bone portions at the interface in response to a compressive force established by the implant.

In any implementations, the aperture may be a slot.

In any implementations, the compression spacer may be metallic.

In any implementations, the compression spacer may include a handle for removing the compression spacer from the gap.

In any implementations, the compression spacer may include a first set of protrusions receivable in a first set of openings in the first bone portion and a second set of protrusions receivable in a second set of openings in the second bone portion to limit relative movement between the first and second bone portions.

A system for an orthopaedic procedure according to an implementation may include a physical anatomical model representative of anatomy. The physical anatomical model may include a first bone portion and a second bone portion. A compression spacer may be dimensioned to separate the first bone portion and the second bone portion under a compressive load, such that removal of the compression spacer may cause movement between the first bone portion and the second bone portion.

In any implementations, a thickness of the compression spacer may be associated with a preselected compressive force such that removal of the compression spacer may cause abutment between the first bone portion and the second bone portion in response to the compressive load being equal to or exceeding the preselected compressive force.

In any implementations, the compression spacer may include an aperture dimensioned to receive an implant that may extend through the first bone portion and the second bone portion.

In any implementations, the first bone portion and the second bone portion may be associated with a common bone of the anatomy.

In any implementations, the first bone portion and the second bone portion may be associated with adjacent bones that establish a joint of the anatomy.

In any implementations, a periphery of the compression spacer may be dimensioned to substantially match a contour of the anatomy.

A method of rehearsing for a surgical procedure according to an implementation may include accessing a virtual anatomical model associated with an anatomy of a patient. The method may include generating a configuration associated with a physical anatomical model that may be representative of the virtual anatomical model. The method may include forming the physical anatomical model. The physical anatomical model may include a body that may be associated with an anatomical profile of a bone. The body may include a first bone portion and a second bone portion. The method may include forming a compression spacer that may be insertable between the first bone portion and the second bone portion.

In any implementations, the method may include inserting an orthopaedic implant through the first bone portion and the second bone portion.

In any implementations, the method may include, after inserting the orthopaedic implant, removing the compression spacer from a gap between the first bone portion and the second bone portion.

In any implementations, the method may include evaluating compression of the orthopaedic implant by determining whether the first bone portion abuts the second bone portion after removing the compression spacer.

In any implementations, the forming step may include printing layers of material on each other to establish the physical anatomical model.

In any implementations, the method may include forming an internal cavity in the first bone portion and the second bone portion. The method may include inserting an orthopaedic implant through the internal cavity to secure the first and second bone portions to each other.

In any implementations, the method may include inserting an orthopaedic implant through an aperture in the compression spacer to secure the first and second bone portions to each other.

In any implementations, the forming step may include printing layers of material on each other to establish the physical anatomical model.

In any implementations, the first bone portion and the second bone portion may be associated with a common bone of the anatomy.

In any implementations, the first bone portion and the second bone portion may be associated with adjacent bones that establish a joint of the anatomy.

In any implementations, the first bone portion may be associated with a long bone of the anatomy.

In any implementations, the method may include inserting an orthopaedic implant through the first bone portion, the second bone portion, and a third bone portion. The first and second bone portions may be associated with a tibia of the anatomy. The third bone portion may be associated with a talus of the anatomy.

A compression spacer for a surgical procedure according to an implementation may include a spacer body configured to be received between first and second bone portions of a physical anatomical model. The spacer body may be dimensioned to have a thickness associated with a compressive force of an implant associated with abutment of the first and second bone portions.

In any implementations, the spacer body may include an aperture dimensioned to receive an implant extending through the first and second bone portions.

In any implementations, the spacer body may include a U-shaped profile.

In any implementations, one or more protrusions may extend from the spacer body and may be dimensioned to be received in one or more openings of one or more of the first and second bone portions.

In any implementations, a handle may extend from the spacer body for manipulation of the compression spacer.

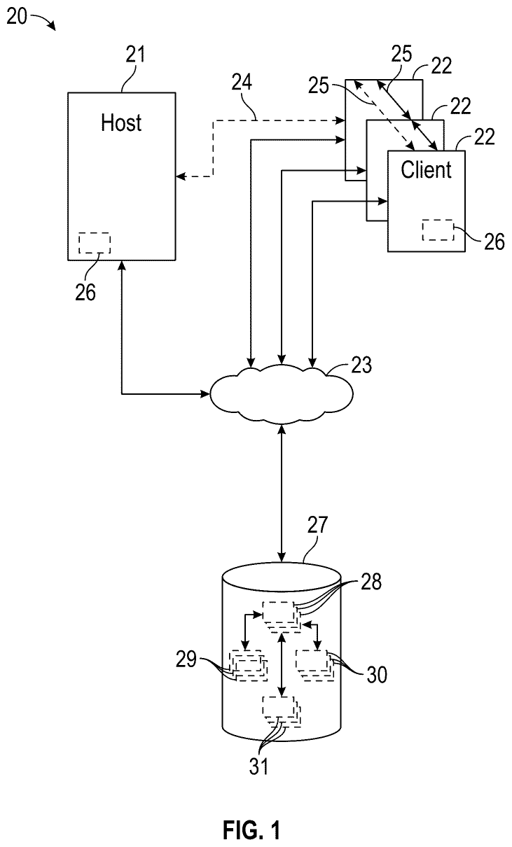

FIG. 1 discloses a planning system 20 that may be utilized for planning surgical procedures. The system 20 may be used for planning orthopaedic procedures, including pre-operatively, intra-operatively and/or post-operatively to create, edit, execute and/or review surgical plans.

The system 20 may include a host computer 21 and one or more client computers 22. The host computer 21 may be configured to execute one or more software programs. In some implementations, the host computer 21 is more than one computer jointly configured to process software instructions serially or in parallel.

The host computer 21 may be in communication with one or more networks such as a network 23 comprised of one or more computing devices. The network 23 may be a private local area network (LAN), a private wide area network (WAN), the Internet, or a mesh network, for example.

The host computer 21 and each client computer 22 may include one or more of a computer processor, memory, storage means, network device and input and/or output devices and/or interfaces. The input devices may include a keyboard, mouse, etc. The output device may include a monitor, speakers, printers, etc. The memory may, for example, include UVPROM, EEPROM, FLASH, RAM, ROM, DVD, CD, a hard drive, or other computer readable medium which may store data and/or other information relating to the planning techniques disclosed herein. The host computer 21 and each client computer 22 may be a desktop computer, laptop computer, smart phone, tablet, or any other computing device. The interface may facilitate communication with the other systems and/or components of the network 23.

Each client computer 22 may be configured to communicate with the host computer 21 directly via a direct client interface 24 or over the network 23. The client computers 22 may be configured to execute one or more software programs, such as a various surgical tools. The planning package may be configured to communicate with the host computer 21 either over the network 23 or directly through the direct client interface 24. In another implementation, the client computers 22 are configured to communicate with each other directly via a peer-to-peer interface 25.

Each client computer 22 may be operable to access and locally and/or remotely execute a planning environment 26. The planning environment 26 may be a standalone software package or may be incorporated into another surgical tool. The planning environment 26 may provide a display or visualization of one or more bone models and related images and one or more implant models via one or more graphical user interfaces (GUI). Each bone model, implant model, and related images and other information may be stored in one or more files or records according to a specified data structure.

The system 20 may include at least one storage system 27, which may be operable to store or otherwise provide data to other computing devices. The storage system 27 may be a storage area network device (SAN) configured to communicate with the host computer 21 and/or the client computers 22 over the network 23. In implementations, the storage system 27 may be incorporated within or directly coupled to the host computer 21 and/or client computers 22. The storage system 27 may be configured to store one or more of computer software instructions, data, database files, configuration information, etc.

In implementations, the system 20 may be a client-server architecture configured to execute computer software on the host computer 21, which may be accessible by the client computers 22 using either a thin client application or a web browser executing on the client computers 22. The host computer 21 may load the computer software instructions from local storage, or from the storage system 27, into memory and may execute the computer software using the one or more computer processors.

The system 20 may include one or more databases 28. The databases 28 may be stored at a central location, such as the storage system 27. In another implementation, one or more databases 28 may be stored at the host computer 21 and/or may be a distributed database provided by one or more of the client computers 22. Each database 28 may be a relational database configured to associate one or more bone models 29 and one or more implant models 30 to each other and/or a surgical plan 31. Each surgical plan 31 may be associated with a respective patient. Each bone model 29, implant model 30 and surgical plan 31 may be assigned a unique identifier or database entry. The database 28 may be configured to store data corresponding to the bone models 29, implant models 30 and surgical plans 31 in one or more database records or entries, and/or may be configured to link or otherwise associate one or more files corresponding to each respective bone model 29, implant model 30 and surgical plan 31. Bone models 29 stored in the database(s) 28 may correspond to respective patient anatomies from prior surgical cases, and may be arranged into one or more predefined categories such as sex, age, ethnicity, defect category, procedure type, etc.

Each bone model 29 may include information obtained from one or more medical devices or tools, such as a computerized tomography (CT), magnetic resonance imaging (MRI) machine and/or X-ray machine, that may obtain one or more images of a patient. The bone model 29 may include one or more digital images and/or coordinate information relating to an anatomy of the patient obtained or derived from the medical device(s). Each implant model 30 may include coordinate information associated with a predefined design. The planning environment 26 may incorporate and/or interface with one or more modeling packages, such as a computer aided design (CAD) package, to render the models 29, 30 as two-dimensional (2D) and/or three-dimensional (3D) volumes or constructs.

The predefined design may correspond to one or more components. The implant models 30 may correspond to implants and components of various shapes and sizes. Each implant may include one or more components that may be situated at a surgical site including plates, screws, anchors, and/or grafts. Each implant model 30 may correspond to a single component or may include two or more components that may be configured to establish an assembly. Each bone model 29 and implant model 30 may correspond to 2D and/or 3D geometry, and may be utilized to utilized to generate a wireframe, mesh and/or solid construct in a display.

Each surgical plan 31 may be associated with one or more of the bone models 29 and implant models 30. The surgical plan 31 may include one or more revisions to a bone model 29 and information relating to a position of an implant model 30 relative to the original and/or revised bone model 29. The surgical plan 31 may include coordinate information relating to the revised bone model and a relative position of the implant model 30 in predefined data structure(s). Revisions to each bone model 29 and surgical plan 31 may be stored in the database 28 automatically and/or in response to user interaction with the system 20.

One or more surgeons and other clinical users may be provided with a planning environment 26 via the client computers 22 and may simultaneously access each bone model 29, implant model 30 and surgical plan 31 stored in the database(s) 28. Each user may interact with the planning environment 26 to create, view and/or modify various aspects of the surgical plan 31. Each client computer 22 may be configured to store local instances of the bone models 29, implant models 30 and/or surgical plans 31, which may be synchronized in real-time or periodically with the database(s) 28. The planning environment 26 may be a standalone software package executed on a client computer 22 or may be provided as one or more services executed on the host computer 21

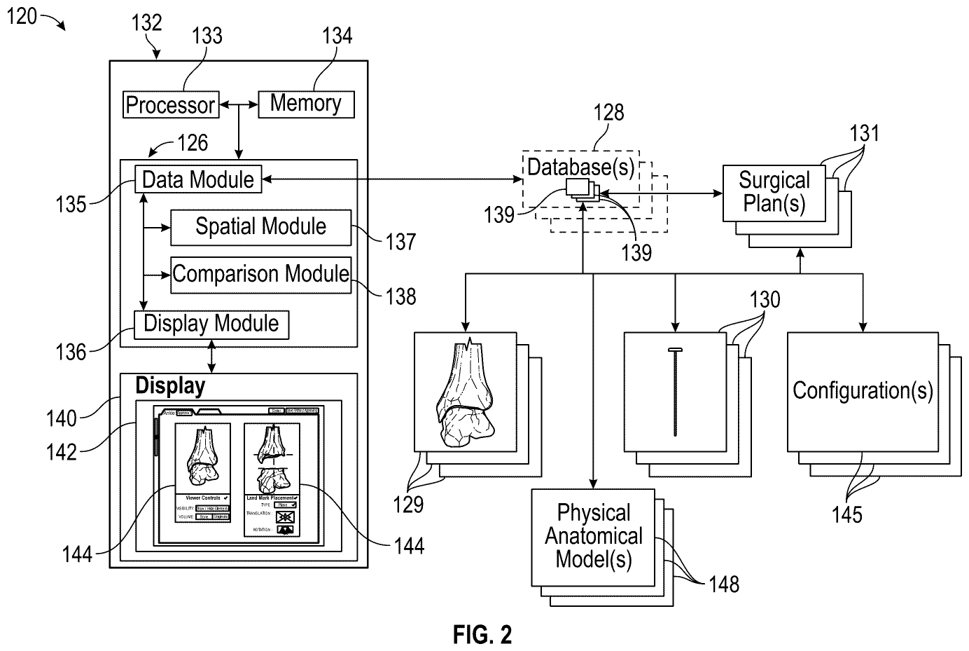

FIG. 2 illustrates a planning system 120 for planning a surgical procedure according to an implementation. The system 120 may be utilized to plan and implement various orthopaedic and other surgical procedures, such as an arthroplasty to repair a joint. The system 120 may be utilized in planning placement of an implant, which may be utilized for securing adjacent bones such as a tibia and talus in an ankle repair. Although the planning systems and methods disclosed herein primarily refer to repair of an ankle joint, it should be understood that the planning system 120 may be utilized in the repair of other locations of the patient and other surgical procedures including repair of other joints such as a shoulder, wrist, hand, hip or knee, and including repair of fractures.

The system 120 may include a computing device 132 including at least one processor 133 coupled to memory 134. The computing device 132 can include any of the computing devices disclosed herein, including the host computer 21 and/or client computer 22 of FIG. 1. The processor 133 may be configured to execute a planning environment 126 for creating, editing, executing and/or reviewing one or more surgical (e.g., pre-operative) plans 131 during pre-operative, intra-operative and/or post-operative phases of a surgery.

The planning environment 126 may include at least a data module 135, a display module 136, a spatial module 137 and a comparison module 138. Although four modules are shown, it should be understood that fewer or more than four modules may be utilized and/or one or more of the modules may be combined to provide the disclosed functionality.

The data module 135 may be configured to access, retrieve and/or store data and other information in the database(s) 128 corresponding to one or more bone model(s) 129, implant model(s) 130 and/or surgical plan(s) 131. The data and other information may be stored in one or more databases 128 as one or more records or entries 139. In implementations, the data and other information may be stored in one or more files that may be accessible by referencing one or more objects or memory locations references by the records or entries 139.

The memory 134 may be configured to access, load, edit and/or store instances of one or more bone models 129, implant models 130 and/or surgical plans 131 in response to one or more commands from the data module 135. The data module 135 may be configured to cause the memory 134 to store a local instance of the bone model(s) 129, implant model(s) 130 and/or surgical plan(s) 131 which may be synchronized with records 139 in the database(s) 128.

The display module 136 may be configured to display data and other information relating to one or more surgical plans 131 in at least one graphical user interface (GUI) 142. The computing device 132 may be coupled to a display device 140. The display module 136 may be configured to cause the display device 140 to display information in the user interface 142. A surgeon or other user may interact with the user interface 142 via the planning environment 126 to create, edit, execute and/or review one or more surgical plans 131.

The system 120 may be configured to generate one or more physical anatomical models 148, including any of the physical anatomical models disclosed herein. The physical anatomical models 148 may be representative of various anatomy and tissue, including any of the bones and joints disclosed herein. The surgeon may perform one or more modifications to the physical anatomical model 148 to rehearse or train for a surgical procedure. The system 120 may be configured to generate configuration(s) 145 associated with respective physical anatomical model(s) 148. The configuration 145 may be utilized in the formation of a physical anatomical model 148. Each physical anatomical model 148 may be representative of, or may otherwise be associated with, a virtual anatomical model 129, including a substantially or generally corresponding geometry, texture, density, porosity, color, etc. as the virtual anatomical model 129. The virtual anatomical model 129 may be associated with an anatomy, such as the anatomy of a patient and/or a hypothetical anatomy. The anatomical models 129 may include one or more anatomical features. The anatomical features may be representative of anatomy, including one or more bones including cartilage, cortical and/or cancellous bone tissue, soft tissue including muscle, ligaments and/or tendons, etc., and/or other tissue. For the purposes of this disclosure, the term “substantially” means±10 percent of the stated value or relationship unless otherwise indicated.

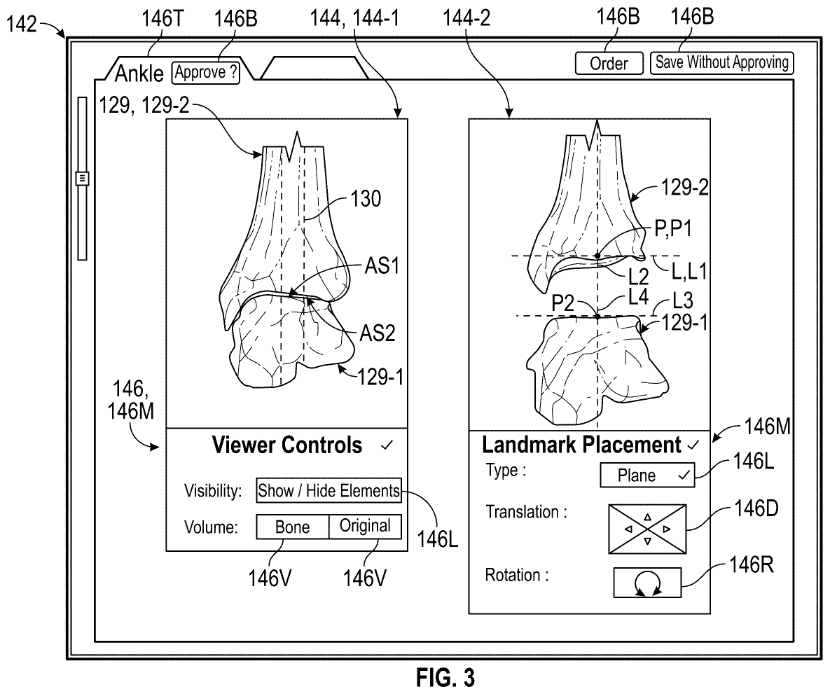

Referring to FIG. 3, with continuing reference to FIG. 2, the user interface 142 may include one or more display windows 144 and one or more objects 146. The objects 146 may include graphics such as menus, tabs and buttons accessible by user interaction. Geometric objects including selected bone model(s) 129 and implant model(s) 130 and other information relating to the surgical plan 131 may be displayed in one or more of the display windows 144.

The implant model 130 may include one or more components associated with an implant. The implant model 130 may be representative of various implants including one or more components that may be situated at a surgical site including plates, anchors, screws, nails, suture, grafts, etc. In implementations, the implant model 130 may be an intramedullary nail or rod, which may be used in a tibio-talo-calcaneal (TTC) fusion procedure. In implementations, the implant model 130 may be associated with an implant suitable to establish a compressive force at an interface between two or more bone portions, such as between a tibia and talus and/or between the talus and calcaneus of a patient.

The display module 136 may be configured to display one or more selected bone models 129 and/or one or more selected implant models 130 in the display windows 144. The display module 136 may be configured such that the selected bone model 129 and/or selected implant model 130 may be selectively displayed and hidden (e.g., toggled) in one or more of the display windows 144 in response to user interaction with the user interface 142, which may provide the surgeon with enhanced flexibility in reviewing aspects of the surgical plan 131.

The data module 135 may be configured to access a first bone model 129-1 and a second bone model 129-2 from the database 128, which may occur automatically or in response to user interaction with the user interface 142. The data module 135 may be configured to store an instance of the first bone model 129-1 and second bone model 129-2 in the memory 134. The first bone model 129-1 and second bone model 129-2 may be associated with a joint. In implementations, one of the bone models 129 may be associated with a long bone such as a tibia, and another one of the bone models 129 may be associated with an adjacent bone such as a talus that may cooperate to establish an ankle or another joint of a patient. In the implementation of FIG. 3, the first bone model 129-1 may be associated with a talus, and the second bone model 129-2 may be associated with a tibia. The display module 136 may be configured to display the first bone model 129-1 and second bone model 129-2 in at least one of the display windows 144 of the user interface 142.

The display windows 144 may include first and second display windows 144-1, 144-2. Although a particular number of display windows 144 are disclosed, the user interface 142 may be configured with any number of display windows 144 in accordance with the teachings disclosed herein. The display windows 144-1, 144-2 may be configured to display a two-dimensional (2D) and/or three-dimensional (3D) representation of the selected bone models 129.

The first display window 144-1 may be configured to display the first bone model 129-1 and second bone model 129-2 relative to each other. The spatial module 137 may be configured to position the bone models 129-1, 129-2 into contact with each other at a specified or defined position and orientation, which may be according to user interaction with the window 144-1, menu 146M, and/or other objects 146 of the user interface 142.

The surgeon or assistant may interact with the display window 144-1 or another portion of the user interface 142 to move the selected bone model 129 and/or selected implant model 130 in 2D space (e.g., up, down, left, right) and/or 3D space (e.g., rotation, tilt, zoom, etc.), which may occur in response to interaction with the directional indicators 146D, 146R.

The second display window 144-2 may be configured to display the first bone model 129-1 and second bone model 129-2 in spaced relationship relative to each other. The surgeon or assistant may interact with the second display window 144-2 or another portion of the user interface 142 to associate one or more landmarks L with the selected bone models 129. The landmarks L may include one or more points P along the anatomy (e.g., P1-P2) and one or more planes (e.g., L1-L4). Exemplary landmarks include a tibial axis, sagittal plane, coronal plane and transverse plane. In implementations, the spatial module 137 may be configured to determine one or more landmarks L based on evaluating a profile of the selected bone model 129. The profile can be compared to one or more profiles of representative bones in the database 128.

The display module 136 may be configured to display one or more implant models 130 in one or more of the display window(s) 144 of the user interface 142. The spatial module 137 may be configured to position the implant model(s) 130 relative to the first and second bone models 129-1, 129-2 automatically and/or in response to user interaction with the user interface 142. The surgeon or other user may select an implant model 130 and a placement of an implant model 130 within the first and second bone models 129-1, 129-2. The surgeon or other user may determine a desired compressive force associated with placement of the implant model 130 relative to the bone models 129-1, 129-2.

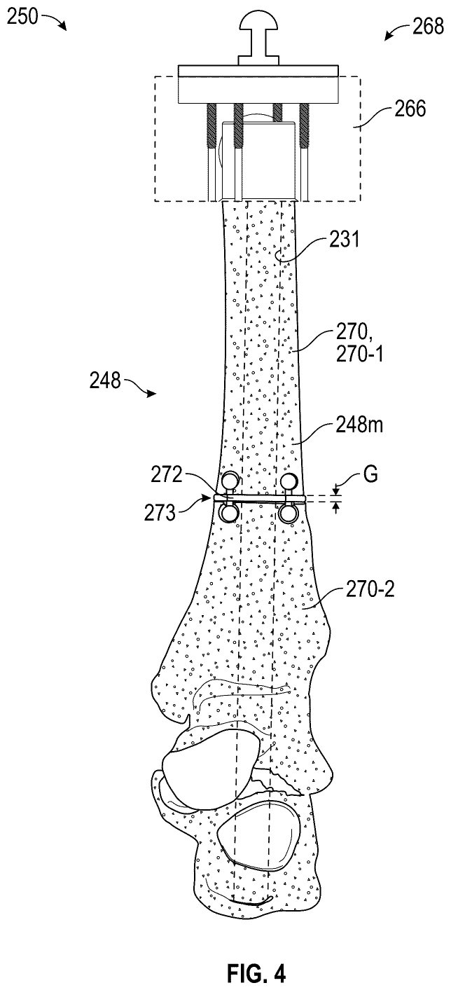

FIG. 4 discloses a system 250 for a surgical procedure according to an implementation. The system 250 may incorporate any of the features disclosed herein. The system 250 may include a physical anatomical model 248. The physical anatomical model 248 may be established based on a configuration 145 (FIG. 2). The physical anatomical model 248 may be secured to at least one fixture 266 to establish an assembly 268. The fixture 266 may be securable to a static structure. The physical anatomical model 248 may include a main body 248M representative of anatomy, including the anatomy of a patient or a hypothetical anatomy. In implementations, the main body 248M may be associated with an anatomical profile of one or more bones and/or joints, including any of the bones and/or joints disclosed herein. In implementations, the bone may be a long bone, such as a fibula, tibia, femur or humerus.

The main body 248M may include two or more (e.g., adjacent) bone portions 270. The bone portions 270 may include a first bone portion 270-1 and a second bone portion 270-2. The first bone portion 270-1 and the second bone portion 270-2 may be associated with a common bone of the anatomy or may be associated with adjacent bones that may establish a joint of the anatomy. In implementations, one or both of the first bone portion 270-1 and the second bone portion 270-2 may be associated with a long bone of the anatomy, such as a tibia.

The system 250 may include at least one compression spacer 272. The compression spacer 272 may be positioned to establish a gap G at an interface 273 between the first bone portion 270-1 and the second bone portion 270-2. The compression spacer 272 may be associated with a preselected compressive force relating to the gap G such that removal of the compression spacer 272 may cause abutment between the first bone portion 270-1 and the second bone portion 270-2 at the interface 273. The compression spacer 272 may be formed from various metallic and/or non-metallic materials, such as stainless steel or titanium. In implementations, the compression spacer 272 may be a different material from one, or both, of the first bone portion 270-1 and the second bone portion 270-2.

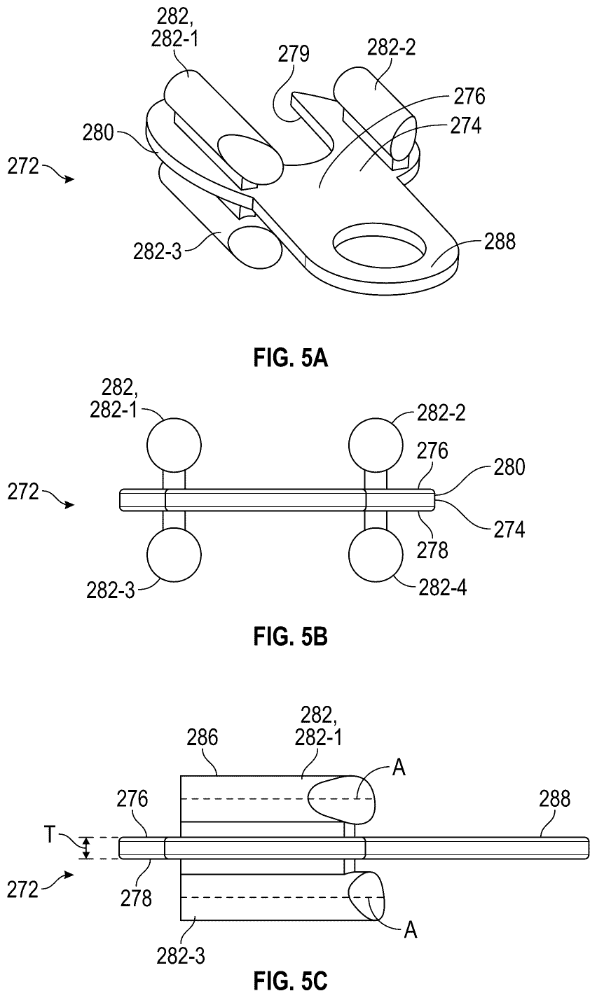

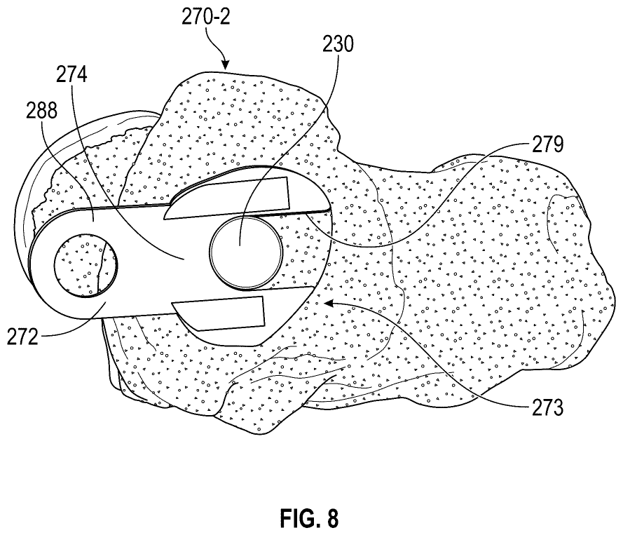

Referring to FIGS. 5A-5C, with continuing reference to FIG. 4, the compression spacer 272 may include a spacer body 274 extending between a first surface 276 and a second surface 278. The first surface 276 may be dimensioned to abut the first bone portion 270-1 (FIG. 4). The second surface 278 may be dimensioned to abut the second bone portion 270-2 (FIG. 4). The spacer body 274 may include a periphery 280 adjoining the first surface 276 and the second surface 278. The periphery 280 of the spacer body 274 may have a contour dimensioned to substantially match a bone contour of one, or both, of the first bone portion 270-1 and the second bone portion 270-2. The spacer body 274 may include a generally U-shaped profile (e.g., FIGS. 5A and 8). The spacer body 274 may define an aperture 279. In implementations, the aperture 279 may be a slot. The aperture 279 may be dimensioned to allow passage of an implant 230 through the interface 273 (e.g., FIG. 8).

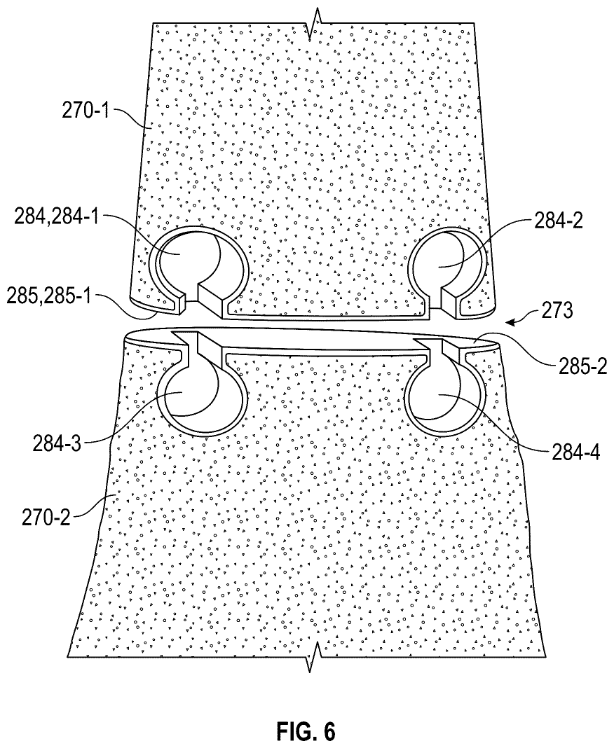

The compression spacer 272 may cooperate with the first and second bone portions 270-1, 270-2 to selectively establish a lock mechanism for limiting relative movement. The compression spacer 272 may include one or more protrusions (e.g., keys) 282 (indicated at 282-1, 282-2, 282-3, 282-4) receivable in corresponding openings (e.g., keyways) 284 (indicated at 284-1, 284-2, 284-3, 284-4 in FIG. 6) in the first bone portion 270-1 and second bone portion 270-2 to limit relative movement between the compression spacer 272 and at least one, or both, of the first bone portion 270-1 and second bone portion 270-2 when in an assembled position. Although four protrusions 282-1, 282-2, 282-3, 282-4 are shown, fewer or more than four protrusions and associated openings may be utilized in accordance with the teachings disclosed herein.

In implementations, the protrusions 282-1, 282-2 may be on an opposite side of the spacer body 274 as the protrusions 282-3, 282-4. The protrusion 282-1 may be opposite the aperture 279 from the protrusion 282-2. The protrusion 282-3 may be opposite the aperture 279 from the protrusion 282-4. The protrusions 282-1, 282-2 may extend from the surface 276. The protrusions 282-3, 282-4 may extend from the surface 278. One or more of the protrusions 282-1, 282-2, 282-3, 282-4 may include a cylindrical body 286 having a longitudinal axis A. The longitudinal axis A may be substantially parallel (±10 degrees) to one or both of the surfaces 276, 278.

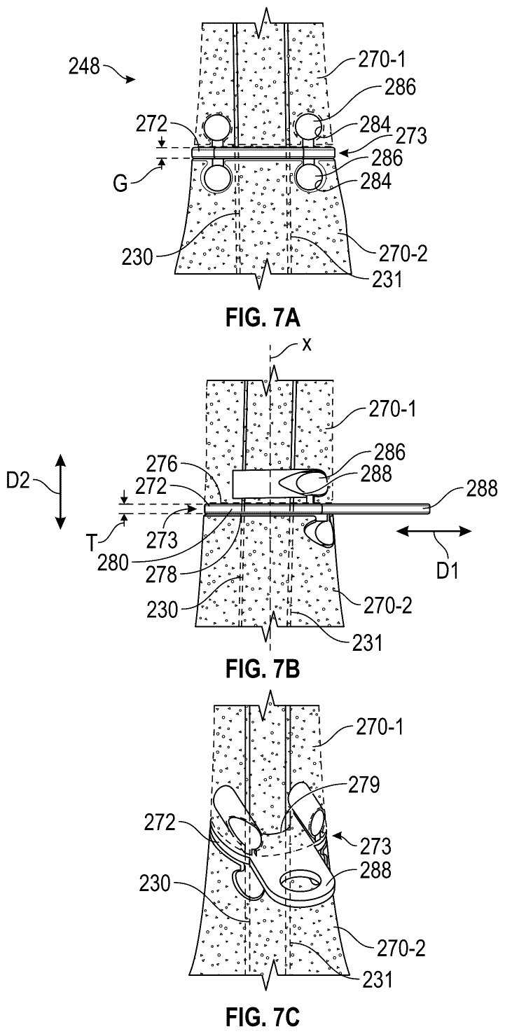

Referring to FIG. 6, with continuing reference to FIGS. 4 and 5A-5C, the openings 284-1, 284-2 may be provided in the first bone portion 270-1. The openings 284-3, 284-4 may be provided in the second bone portion 270-2. The cylindrical body 286 may be dimensioned to mate with the respective opening 284 to limit relative movement (e.g., FIG. 7A).

One, or both, of the first bone portion 270-1 and the second bone portion 270-2 may include a reinforcement member 285 (indicated at 285-1 and 285-2) at the interface 273. A first reinforcement member 285-1 may be provided on the first bone portion 270-1. A second reinforcement member 285-2 may be provided on the second bone portion 270-2. In implementations, the reinforcement members 285 may a material that may be the same or may differ from a material of the first bone portion 270-1 and/or the second bone portion 270-2. In implementations, the reinforcement members 285 include a metallic material, including any of the materials disclosed herein. The reinforcement members 285 may include a material that may be the same or may different from a material of the compression spacer 272. The reinforcement members 285 may be dimensioned to abut the compression spacer 272 when the compression spacer 272 is received at the interface 273. The reinforcement members 285 may establish the openings 284. In implementations, the reinforcement members 285-1, 285-2 may abut one another after removal of the compression spacer 272 if a sufficient (e.g., preselected) compressive force is achieved. The reinforcement members 285 may serve to reinforce the first and/or second bone portions 270-1, 270-2 adjacent to the interface 273. The reinforcement members 285 may also serve to more uniformly spread compressive loads applied to the first and/or second bone portions 270-1, 270-2, which may include a relatively softer (e.g., non-metallic) material than a (e.g., metallic) material of the reinforcement members 285 and/or compression spacer 272. In other implementations, the reinforcement members 285 may be omitted.

Referring to FIGS. 7A-7C, with continuing reference to FIGS. 4, 5A-5C and 6, the compression spacer 272 may be received at the interface 273 between the first bone portion 270-1 and the second bone portion 270-2. An implant 230 may be at least partially received in the model 248 (shown in dashed lines). The implant 230 may extend at least partially through the first bone portion 270-1 and/or the second bone portion 270-2. The implant 230 may be adapted to establish a compressive force between the first bone portion 270-1 and the second bone portion 270-2. The implant 230 may be any of the implementations discussed herein, such as an intramedullary nail. The intramedullary nail may be positionable in an intramedullary canal of the model 248. The compression spacer 272 may be removable to evaluate compression between the first bone portion 270-1 and the second bone portion 270-2 at the interface 273 in response to a compressive force established by the implant 230. The compression may be associated with a compressive force specified in a surgical plan 131 (FIG. 2). The implant 230 may extend through the aperture 279 (e.g., FIGS. 5A and 8). A (e.g., reaming or drill) path 231 may be established in one, or both, of the first bone portion 270-1 and the second bone portion 270-2. The reaming path 231 may be dimensioned to at least partially receive the implant 230. The reaming path 231 may be formed with the physical anatomical model 248 utilizing any of the techniques disclosed herein, such as through rapid prototyping (e.g., printing) and other additive manufacturing techniques. In other implementations, the reaming path 231 may be established subsequent to forming the physical anatomical model 248. The reaming path 231 may be established using a machining or other material removal technique. The surgeon or other clinical user may utilize a surgical instrument such as a drill to establish the reaming path 231.

The compression spacer 272 may include a (e.g., pull-out) handle 288 for manipulating the compression spacer 272 by the surgeon or clinical user. The handle 288 may be useful for moving the compression spacer 272 into and/or out of the gap G at the interface 283 (e.g., FIG. 7A). With reference to FIGS. 5A, 7B and 8, the handle 288 may extend from the spacer body 274. The aperture 279 may be open to the periphery 280 to allow the compression spacer 272 to be removed from the interface 283 while the implant 230 is received through the interface 273. In the implementation of FIG. 7B, the protrusions 282 and openings 284 may be dimensioned such that the compression spacer 272 may be moveable in a first direction D1. The first direction D1 may be substantially perpendicular relative to a (e.g., longitudinal) axis X of the model 248 at the interface 273. The protrusions 282 and openings 284 may be dimensioned to limit movement in a second direction D2 between the compression spacer 272 and the first and/or second bone portions 270-1, 270-2. The second direction D2 may be substantially perpendicular or otherwise transverse to the first direction D1. In implementations, the second direction D2 may be substantially parallel to the axis X.

The compression spacer 272 may be dimensioned to separate the first bone portion 270-1 and the second bone portion 270-2 under a compressive load, which may be established by the implant 230, such that removal of the compression spacer 272 may cause movement between the first bone portion 270-1 and the second bone portion 270-2. In implementations, a thickness T of the compression spacer 272 (FIG. 7B) may be associated with a preselected compressive force, such as a (e.g., preselected) compressive force of the implant 230, such that removal of the compression spacer 272 may cause abutment between the first bone portion 270-1 and the second bone portion 270-2 in response to the compressive load being equal to or exceeding the preselected compressive force. The preselected compressive force may be specified in a surgical plan 131 (FIG. 2). A thickness T of the spacer body 274 may be the distance between the surfaces 276, 278 (e.g., FIGS. 5C and 7B). As the thickness T increases, a greater compressive force may be needed to cause abutment between the first bone portion 270-1 and the second bone portion 270-2 when the compressive spacer 272 is removed. In implementations, the thickness T may therefore be preselected in order to evaluate the compressive force of an implant 230 selection and/or configuration. In implementations, a set of compression spacers 272 of various dimensions (e.g., thicknesses) may be provided with the physical anatomical model 248 as a kit. The surgeon or clinical user may select one or more compression spacers 272 from the kit to evaluate the compression associated with a selected implant 230 that may secure the two portions 270-1, 270-2 to each other. The set of compression spacers 272 may include incremental thicknesses and/or a preselected thickness associated with a surgical plan 131 (FIG. 2).

FIG. 8 discloses a cross sectional view through the interface 273. The aperture 279 may be dimensioned to allow the implant 230 to extend through the compression spacer 272. The implant 230 may be dimensioned to pass through the aperture 279 for securing the first and second bone portions 270-1, 270-2 each other (e.g., FIGS. 7A-7C). The compression spacer 272 may be removable from the physical anatomical model 248 when the first and second bone portions 270-1, 270-2 are secured together.

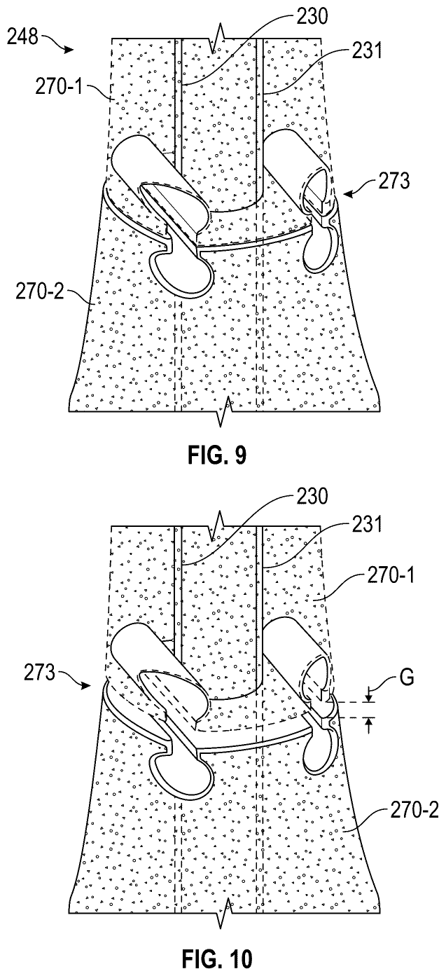

FIG. 9 discloses the physical anatomical model 248 after the compression spacer 272 (not shown) is removed and sufficient compressive force is achieved. The implant 230 has achieved sufficient compressive force, such that the first bone portion 270-1 and the second bone portion 270-2 are in abutment at the interface 273.

FIG. 10 discloses the physical anatomical model 248 after the compression spacer 272 (not shown) is removed and insufficient compressive force is achieved. The implant 230 has not achieved sufficient compressive force, such that the first bone portion 270-1 and the second bone portion 270-2 are spaced from one another and not in abutment. There remains a gap G after removal of the compression spacer 272. The gap G may be equal to or less than the thickness T of the compression spacer 272 (FIG. 7B).

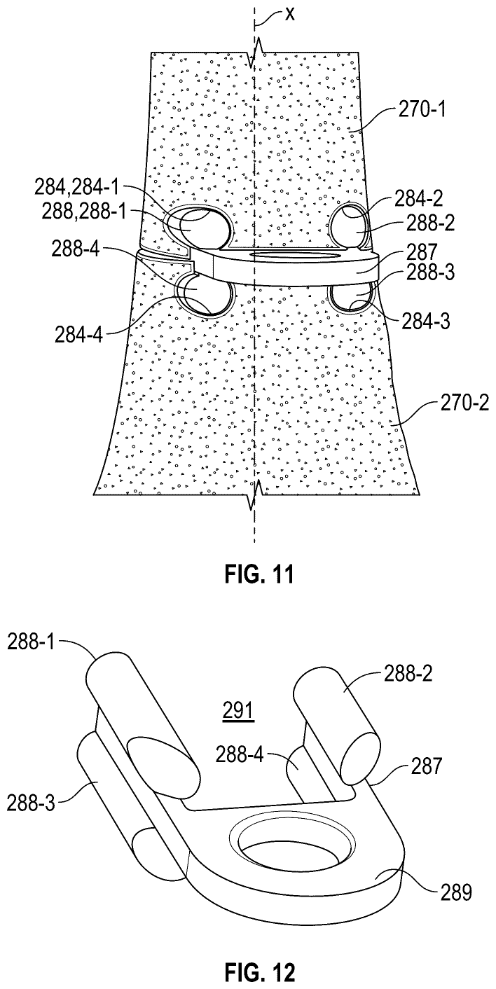

Referring to FIGS. 11 and 12, after the compression spacer 272 (not shown) is removed and sufficient compressive force is achieved, a locking key 287 may be inserted into the openings 284 to limit relative rotation, such as rotation about the axis x, of the first bone portion 270-1 and the second bone portion 270-2. The locking key 287 may limit relative axial movement of the first bone portion 270-1 and the second bone portion 270-2 along the axis x. A number of locking protrusions 288 (indicated at 288-1, 288-2, 288-3, 288-4) are dimensioned to be received within the openings 284. The locking protrusion 288-1 may be received within the opening 284-1, the locking protrusion 288-2 may be received within the opening 284-2, the locking protrusion 288-3 may be received within the opening 284-3, and locking protrusion 288-4 may be received within the opening 284-4. The locking protrusions 288 may be shaped similarly to the protrusions 286 of the compression spacer 272. The locking protrusions may include cylindrical bodies dimensioned to mate with the respective opening 284. In implementations, there may be one locking protrusion 288 per opening 284. The locking protrusions 288 may be spaced from a handle 289 for insertion and removal of the locking key 287. In implementations, the locking key 287 may be configured substantially similarly to an associated compression spacer 272, but the locking key 287 does not have a spacer body 274 (see FIGS. 5A-5C).

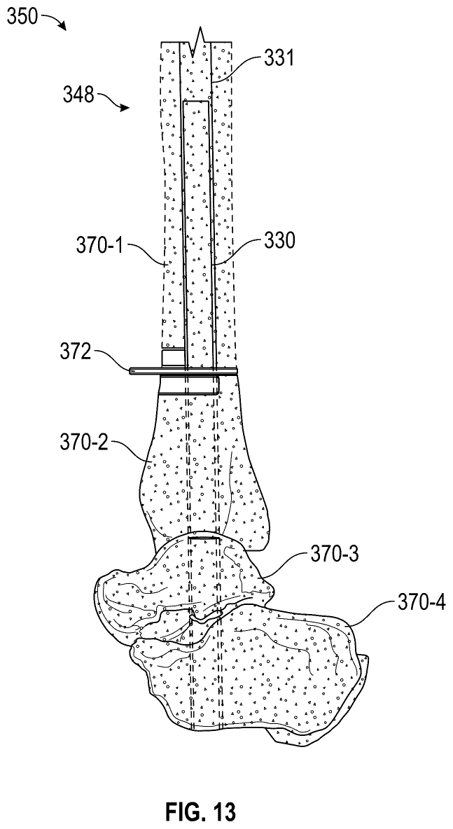

FIG. 13 discloses a system 350 including physical anatomical model 348 and a compression spacer 372. In implementations, the compression spacer 372 may act as a spacer to hold the proximal and distal portions 370-1, 370-2 of a model tibia together for an implant 330 installation for a TTC fusion procedure. The implant 330 may be a hindfoot nail for a TTC fusion procedure. In implementations, the compression spacer 372 may be dimensioned to allow the implant 330 to compress a distance (e.g., 2 mm) after removal of the compression spacer 372 from the physical anatomical model 348. The implant 330 may extend through a third bone portion 370-3. The third bone portion 370-3 may be representative of a talus. The implant 330 may extend through a fourth bone portion 370-4. The fourth bone portion 370-4 may be representative of a calcaneus.

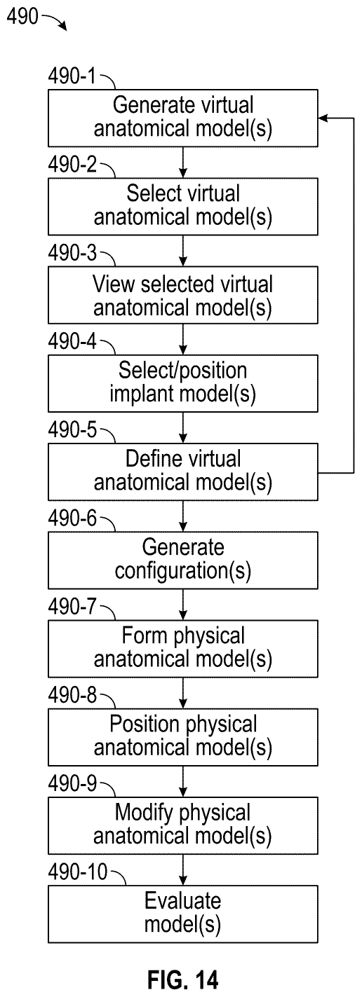

FIG. 14 discloses a method of planning and performing a surgical procedure in a flowchart 490 according to an implementation. The method 490 may be utilized to pre-operatively plan, rehearse and/or train for various surgical procedures. The method 490 may be utilized with any of the planning systems and virtual and physical anatomical models disclosed herein. The method 490 may be utilized to evaluate the accuracy in which a surgeon implements a surgical procedure on a physical anatomical model associated with an anatomy of a patient. The method 490 may be utilized to evaluate compression between two or more portions of a physical anatomical model, which may be representative of portions of the same bone or two or more different (e.g., adjacent) bones. The method 490 may utilize one or more compression spacers to evaluate the compression, which may be established by a respective orthopaedic implant. Fewer or additional steps than are recited below could be performed within the scope of this disclosure, and the recited order of steps is not intended to limit this disclosure.

Referring to FIG. 2, with continuing reference to FIG. 14, at step 490-1, one or more virtual anatomical models 129 may be generated or otherwise defined. Each virtual anatomical model 129 may be associated with an anatomy of a patient and may be generated utilizing any of the techniques disclosed herein.

At step 490-2, one or more virtual anatomical models 129 may be selected from a set of virtual anatomical models 129. The virtual anatomical models 129 may be stored in memory of a computing device, such as in the database 128 or the memory 134 of the computing device 132. Selecting the virtual anatomical model 129 may include selecting from various parameters associated with the set of virtual anatomical models 129. The parameters may include any of the parameters disclosed herein, including patient classification, anatomy and/or defect. The parameters may be selected in response to user interaction with the graphical user interface 142. The virtual anatomical models 129 may include any of the anatomies and tissue types disclosed herein, including bone, ligament, tendon, cartilage, etc. At step 490-3, the selected virtual anatomical model(s) 129 may be viewed in the graphical user interface 142.

At step 490-4, one or more implant models 130 may be selected and/or positioned relative to the selected virtual anatomical model(s) 129. Each implant model 130 may be selected from a set of implant models 130. The implant models 130 may be stored in memory of a computing device, such as in the database 128 or the memory 134 of the computing device 132. The implant models 130 may be associated with any of the implants disclosed herein.

At step 490-5, aspects of one or more of the virtual anatomical models 129 may be defined. Each virtual anatomical model 129 may be defined prior, during and/or subsequent to generating the virtual anatomical model 129 at step 490-1 and/or selecting the virtual anatomical model 129 at step 490-2. Defining the virtual anatomical model 129 may include setting one or more parameters of the virtual anatomical model 129, including any of the parameters disclosed herein. The parameters may be selected in response to user interaction with the graphical user interface 142. The parameters may be associated with one or more indicators.

At step 490-6, one or more configurations (e.g., definitions) 145 may be generated. Each configuration 145 may be associated with a physical anatomical model 148 and may be generated utilizing any of the techniques disclosed herein. The configuration 145 may be representative of the selected virtual anatomical model 129. Each configuration may be generated in response to selecting the respective virtual anatomical model 129 at step 490-2 and/or defining the selected virtual anatomical model 129 at step 490-5. The configuration 145 may be established according to the selection or specification of any parameters associated with the selected virtual anatomical model 129. The configuration 145 may include data and other information sufficient to establish a physical anatomical model 148 based on the parameters of the selected virtual anatomical model 129, including coordinate information, moduli of elasticity and color schemes of the associated tissues, etc.

Referring to FIGS. 4 and 5A-5C, with continuing reference to FIGS. 2 and 14, step 490-6 may include generating a configuration for a compression spacer 272. Dimensions of the spacer 272 may be established based on a (e.g., preselected) compressive force of an implant 230. With reference to FIGS. 5C and 7B, a thickness T of the spacer 272 may be selected based on a (e.g., preselected) compressive force of the implant 230. The spacer 272 may be dimensioned to evaluate compression between two or more adjacent bones, which may establish a joint through which the implant 230 may pass.

At step 490-7, one or more physical anatomical models 248 may be fabricated or otherwise formed based on the generated configuration 145. Each physical anatomical model 248 may be formed utilizing any of the techniques disclosed herein. The physical anatomical model 248 may be a monolithic structure or may have one or more portions releasably secured to each other. The spacer 272 may be fabricated or otherwise formed based on the generated configuration 145.

Various techniques may be utilized to form the physical anatomical models. Each physical anatomical model may be formed utilizing any of the techniques disclosed herein, such as rapid prototyping (e.g., printing) and other additive manufacturing techniques, casting, machining, etc. The physical anatomical model may have a unitary construction or may have two or more components fixedly attached or otherwise secured to each other to establish a unit.

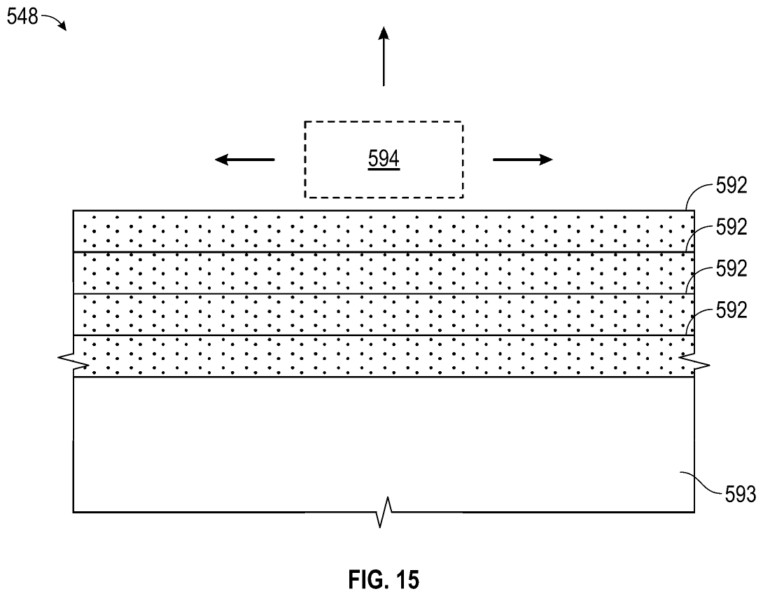

In the implementation of FIG. 15, one or more layers 592 of material may be printed or otherwise formed on a substrate 593 to establish a physical anatomical model 548. The physical anatomical model 548 may be representative of a virtual anatomical model, including any of the virtual anatomical models disclosed herein. A device 594 such as a three-dimensional printer may be configured to form the layers 592 according to data and other information associated with the respective configuration 145. The layers 592 of material may include any of the constructions, materials, color schemes, textures, porosities, etc. disclosed herein. The layers 592 may have respective moduli of elasticity that may substantially correspond to moduli of elasticity of respective biomaterial of the anatomy. The porosities of the material forming the physical anatomical model 548 may substantially approximate the porosity or density of the respective tissue. The physical anatomical model 548 may be formed with materials having different transparencies, translucence, opacity, etc., utilizing any of the techniques disclosed herein.

Step 490-7 may include forming the layers 592 of material to establish the physical anatomical model 548. The layers 592 may be formed concurrently and/or sequentially. Each layer L may be homogenous or heterogenous. Heterogenous layers may incorporate different regions associated with respective tissue types, densities, porosities, colors, etc. Step 490-7 may include printing the layers 592 of material on each other to establish one or more components of the physical anatomical model 548, including any of the components disclosed herein.

Referring to FIG. 4, with continuing reference to FIGS. 2 and 14, at step 490-8 the physical anatomical model 248 may be positioned or otherwise prepared. The physical anatomical model 248 may be secured to one or more fixtures 266 to establish an assembly 268.

At step 490-9, one or more modifications to the physical anatomical model(s) 248 may be performed. Step 490-9 may include removing a portion of the physical anatomical model 248 to establish a revised physical anatomical model 248. Various modifications may be performed to simulate surgical operations performed on an anatomy, including any of the modifications disclosed herein such as one or more incision, cutting, drilling, reaming, resection and implantation operations. Each modification may result in permanently altering a geometry of the physical anatomical model 248.

Referring to FIGS. 4 and 9-10, with continuing reference to FIG. 14, at step 490-10 the physical anatomical model(s) 248 may be evaluated utilizing any of the techniques disclosed herein. Step 490-10 may include position a compression spacer 272 between the first bone portion 270-1 and the second bone portion 270-2 to establish a gap G. The first and/or second bone portions 270-1, 270-2 and compression spacer 272 may be under a compressive load at an interface 273. The compressive load may be established by an orthopaedic implant 230. The compression spacer 272 may be dimensioned to separate the first and second bone portions 270-1. The compression spacer 272 may be selected from a set of compression spacers 272 having various thicknesses, which may be incremental and/or specified in a surgical plan 131 (FIG. 2). Step 490-10 may include removing the compression spacer 272 from the gap G between the first bone portion 270-1 and the second bone portion 270-2, including when the first and/or second bone portions 270-1, 270-2 are under a compressive load. Step 490-10 may include evaluating compression of the implant 230 by determining whether the first bone portion 270-1 abuts the second bone portion 270-2 after removing the compression spacer 272 from between the first and second bone portions 270-1, 270-2. With reference to FIGS. 11 and 12, after removal of the compressor spacer 272, a locking key 287 may be positioned to prevent relative rotation and or axial movement of the first and second bone portions 270-1, 270-2.

The novel devices and methods of this disclosure provide versatility in planning, rehearsing and training for surgical procedures utilizing physical anatomical models. The physical anatomical models may be representative of various anatomy. The surgeon may interact with the disclosed system to gain familiarity with the selected anatomy and various surgical procedures that may be utilized to implement a surgical plan, including testing and evaluating the compression of implants in various orthopaedic procedures.

Although the different examples are illustrated as having specific components, the examples of this disclosure are not limited to those particular combinations. It is possible to use some of the components or features from any of the embodiments in combination with features or components from any of the other embodiments.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. A system for an orthopaedic procedure comprising:
a physical anatomical model including a main body representative of anatomy, wherein the main body includes a first bone portion and a second bone portion; and
a compression spacer positionable to establish a gap at an interface between the first bone portion and the second bone portion, wherein the compression spacer is associated with a preselected compressive force relating to the gap.

2. The system of claim 1, wherein the compression spacer includes a spacer body extending between a first surface and a second surface, the first surface dimensioned to abut the first bone portion, and the second surface dimensioned to abut the second bone portion.

3. The system of claim 2, wherein the spacer body includes a periphery adjoining the first surface and the second surface.

4. The system of claim 3, wherein the periphery of the spacer body has a contour dimensioned to substantially match a bone contour of at least one of the first bone portion and the second bone portion.

5. The system of claim 3, wherein the compression spacer includes a slot dimensioned to allow passage of an implant through the interface.

6. The system of claim 5, wherein the compression spacer includes a first protrusion receivable in a first opening in the first bone portion to limit relative movement between the compression spacer and the first bone portion.

7. The system of claim 6, wherein the first protrusion includes a cylinder having a longitudinal axis substantially parallel with the first surface.

8. (canceled)

9. The system of claim 6, wherein the compression spacer includes a second protrusion receivable in a second opening in the first bone portion, and the first protrusion is opposite the slot from the second protrusion.

10. The system of claim 6, wherein the compression spacer includes a second protrusion receivable in a second opening in the first bone portion, and the first protrusion extends from the first surface, the first opening is established in the first bone portion, the second protrusion extends from the second surface, and the second opening is established in the second bone portion such that the compression spacer limits relative movement between the first and second bone portions at the interface in an assembled position.

11. The system of claim 1, wherein the compression spacer includes a first protrusion receivable in a first opening in the first bone portion to limit relative movement between the compression spacer and the first bone portion.

12. The system of claim 11, wherein the compression spacer includes a second protrusion receivable in a second opening in the second bone portion to limit relative movement between the compression spacer and the second bone portion.

13. The system of claim 1, wherein the compression spacer includes an aperture dimensioned to receive an implant extending through the first bone portion and the second bone portion.

14. The system of claim 13, wherein the implant includes an intramedullary nail.

15. The system of claim 13, wherein the preselected force is selected such that removal of the compression spacer causes the first and second bone portions to abut each other at the interface, and the compression spacer is removable to evaluate compression between the first and second bone portions at the interface in response to a compressive force established by the implant.

16. (canceled)

17. (canceled)

18. The system of claim 1, wherein the compression spacer includes a handle for removing the compression spacer from the gap.

19. The system of claim 1, wherein the compression spacer includes a first set of protrusions receivable in a first set of openings in the first bone portion and a second set of protrusions receivable in a second set of openings in the second bone portion to limit relative movement between the first and second bone portions.

20. A system for an orthopaedic procedure comprising:
a physical anatomical model representative of anatomy, wherein the physical anatomical model includes a first bone portion and a second bone portion; and
a compression spacer dimensioned to separate the first bone portion and the second bone portion under a compressive load such that removal of the compression spacer causes movement between the first bone portion and the second bone portion, and including an aperture dimensioned to receive an implant extending through the first bone portion and the second bone portion, wherein a periphery of the compression spacer is dimensioned to substantially match a contour of the anatomy, and a thickness of the compression spacer is associated with a preselected compressive force such that removal of the compression spacer causes abutment between the first bone portion and the second bone portion in response to the compressive load being equal to or exceeding the preselected compressive force.

21. (canceled)

22. (canceled)

23. (canceled)

24. (canceled)

25. (canceled)

26. A method of rehearsing for a surgical procedure comprising:
accessing a virtual anatomical model associated with an anatomy of a patient;
generating a configuration associated with a physical anatomical model that is representative of the virtual anatomical model;
forming the physical anatomical model, the physical anatomical model including a body associated with an anatomical profile of a bone, the body including a first bone portion and a second bone portion; and
forming a compression spacer insertable between the first bone portion and the second bone portion.

27. The method of claim 26, the method comprising:
inserting an orthopaedic implant through the first bone portion and the second bone portion;
after inserting the orthopaedic implant, removing the compression spacer from a gap between the first bone portion and the second bone portion; and
evaluating compression of the orthopaedic implant by determining whether the first bone portion abuts the second bone portion after removing the compression spacer.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. The system of claim 20, wherein the compression spacer includes a first protrusion receivable in a first opening in the first bone portion to limit relative movement between the compression spacer and the first bone portion, the compression spacer includes a second protrusion receivable in a second opening in the first bone portion, and the first protrusion is opposite the aperture from the second protrusion.
